(19) United States
(12) Patent Application Publication
(10) Pub. No.: US 2004/0259881 A1
(43) Pub. Date: Dec. 23, 2004

(54) Nitrogenous heterocyclic compounds

(76) Inventors: Anjali Pandey, Fremont, CA (US); Robert M Scarborough, Half Moon Bay, CA (US); Kenji Matsuno, Tokyo (JP); Michio Ichimura, Tokyo (JP); Yuji Nomoto, Shizuoka (JP); Shinichi Ide, Tokyo (JP); Eiji Tsukuda, Tokyo (JP); Junko Sasaki, Akita (JP); Shoji Oda, Tokyo (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/344,907

(22) PCT Filed: Aug. 17, 2001
(86) PCT No.: PCT/US01/41749

Related U.S. Application Data
(60) Provisional application No. 60/266,120, filed on Feb. 2, 2001.

(51) Int. Cl. A61K 31/517; C 07D 4 3/14; C 07D 4 3/04

(52) U.S. Cl. 514/252.17; 544/284

(57) ABSTRACT

The present invention relates to nitrogen-containing heterocyclic compounds and pharmaceutically acceptable salts thereof which have inhibitory activity on the phosphorylation of kinases, which inhibits the activity of such kinases. The invention is also related to a method of inhibiting kinases and treating diseases states in a mammal by inhibiting the phosphorylation of kinases. In a particular aspect the present invention provides nitrogen-containing heterocyclic compounds and pharmaceutically acceptable salts thereof which inhibit phosphorylation of PDGF receptor to hinder abnormal cell growth and cell wandering, and a method for preventing or treating cell-proliferative diseases such as arteriosclerosis, vascular reobstruction, cancer and glomerulosclerosis.

### TECHNICAL FIELD

The present invention relates to nitrogen-containing heterocyclic compounds and pharmaceutically acceptable salts thereof which have inhibitory activity on the phosphorylation of kinases, which inhibits the activity of such kinases. The invention is also related to a method of inhibiting kinases and treating disease states in a mammal by inhibiting the phosphorylation of kinases.

### BACKGROUND ART

PDGF (platelet-derived growth factor) is known to act as an aggravating factor for cell-proliferative diseases such as arteriosclerosis, vascular reobstruction after percutaneous coronary angioplasty and bypass operation, cancer, glomerulonephritis, glomerulosclerosis, psoriasis and articular rheumatism [Cell, 46, 155-169 (1986); Science, 253, 1129-1132 (1991); Nippon Rinsho (Japanese J. of Clinical Medicine), 50, 3038-3045 (1992); Nephrol Dial Transplant, 10, 787-795 (1995); Kidney International, 43 (Suppl. 39), 86-89 (1993); Journal of Rheumatology, 21, 1507-1511 (1994); Scandinavian Journal of Immunology, 27, 285-294 (1988), etc.].

As for quinazoline derivatives which are useful as drugs, N,N-dimethyl-4-(6,7-dimethoxy-4-quinazolinyl)-1-piperazine carboxamide is described as a bronchodilator in South African Patent No. 67 06512 (1968). Dimethoxyquinazoline derivatives are described as inhibitors of phosphorylation of epidermal growth factor (EGF) receptor in Japanese Published Unexamined Patent Application No. 208911/93 and WO 96/09294. Quinoline derivatives having benzodiazepin receptor agonist activity are described in Pharmacology Biochemistry and Behavior, 53, 87-97 (1996) and European Journal of Medicinal Chemistry, 31, 417-425 (1996), and quinoline derivatives which are useful as anti-parasite agents are described in Indian Journal of Chemistry, 26B, 550-555 (1987).

Inhibitors of phosphorylation of PDGF receptor so far known include bismono- and bicyclic aryl compounds and heteroaryl compounds (WO 92/20642), quinoxaline derivatives [Cancer Research, 54, 6106 (1994)], pyrimidine derivatives (Japanese Published Unexamined Patent Application No. 87834/94) and dimethoxyquinoline derivatives [Abstracts of the 16th Annual Meeting of the Pharmaceutical Society of Japan (Kanazawa) (1996), 2, p. 275, 29(C2) 15-2].

### DISCLOSURE OF THE INVENTION

An object of the present invention is to provide nitrogen-containing heterocyclic compounds and pharmaceutically acceptable salts thereof which have inhibitory activity on the phosphorylation of kinases, which inhibits the activity of the kinases. Particularly important kinase inhibition according to the invention is of receptor tyrosine kinases including platelet-derived growth factor (PDGF) receptor, Flt3, CSF-1R, epidermal growth factor receptor (EGRF), fibroblast growth factor (FGF), vascular endothelial growth factor receptor (VEGFR) and others. Another class of kinase inhibition according to the invention is inhibitory activity nonreceptor tyrosine kinases including src and abl, and the like. A third class of kinase inhibition according to the invention is inhibitory activity toward serine/threonine kinases, including such kinases as MAPK, MEK and cyclin dependent kinases (CDKs) that mediate cell prolifetation, AKT and CDK such that mediate cell survival and NIK that regulate inflammatory responses. Inhibition of such kinases can be used to treat diseases involving cell survival, proliferation and migration, including cardiovascular disease, such as arteriosclerosis and vascular reobstruction, cancer, glomerulosclerosis fibrotic diseases and inflammation, as well as the general treatment of cell-proliferative diseases.

In a preferred embodiment, the present invention provides compounds and pharmaceutically acceptable salts thereof which inhibit or prevent inhibition of phosphorylation of at least one PDGF receptor by at least one tyrosine kinase. Such PDGF receptor kinase inhibition can hinder abnormal cell growth and cell wandering, and thus such compounds are useful for the prevention or treatment of cell-proliferative diseases such as arteriosclerosis, vascular reobstruction, cancer and glomerulosclerosis.

The present invention relates to nitrogen-containing heterocyclic compounds represented by formula I as follows:

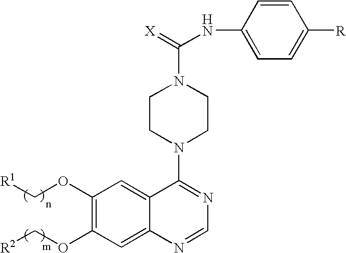

wherein

m and n are each independently from 2 to 8,

X is O or S; and

R is a member selected from the group consisting of:

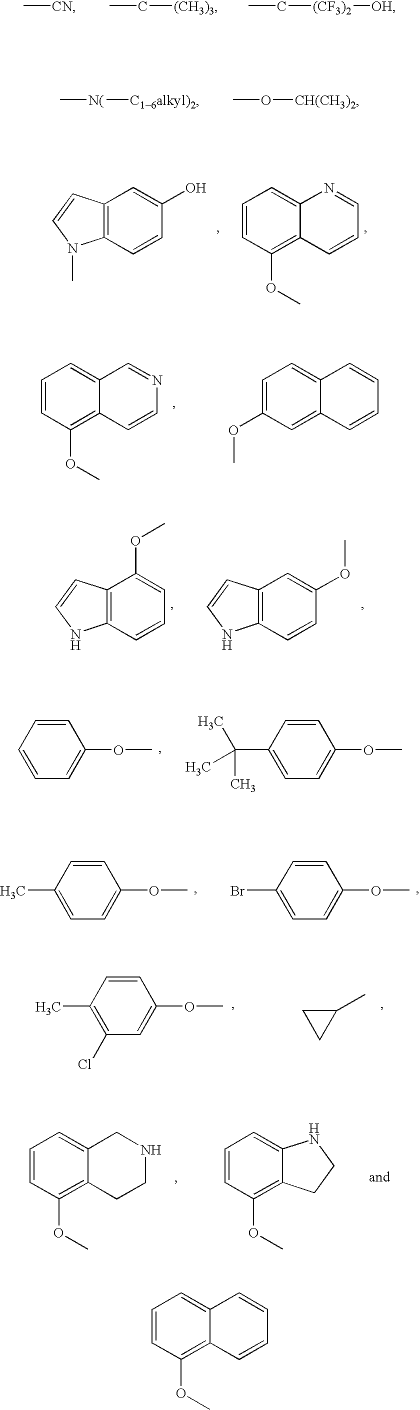

R¹ and R² are each independently a member selected from the group consisting of —OH, —O—CH₃ and

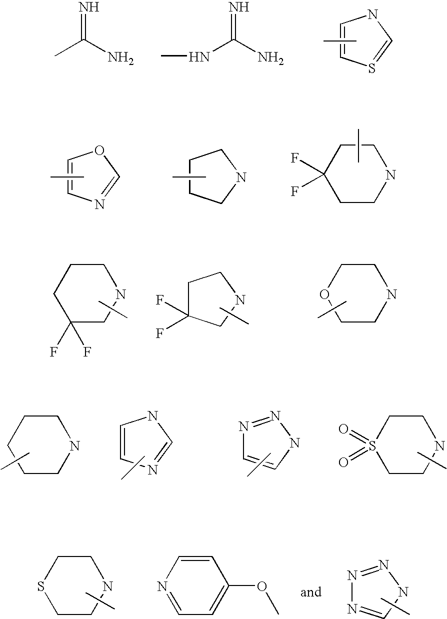

and all pharmaceutically acceptable isomers, salts, hydrates, solvates and prodrug derivatives thereof.

In a preferred aspect, the invention provides such compounds wherein n and m are each independently 2 or 3.

The pharmaceutically acceptable salts of the compounds according to formula (I) include pharmaceutically acceptable acid addition salts, metal salts, ammonium salts, organic amine addition salts, amino acid addition salts, etc. Examples of the pharmaceutically acceptable acid addition salts of the compounds of formula (I) are inorganic acid addition salts such as hydrochloride, sulfate and phosphate, and organic acid addition salts such as acetate, maleate, fumarate, tartrate, citrate and methanesulfonate. Examples of the pharmaceutically acceptable metal salts are alkali metal salts such as sodium salt and potassium salt, alkaline earth metal salts such as magnesium salt and calcium salt, aluminum salt and zinc salt. Examples of the pharmaceutically acceptable ammonium salts are ammonium salt and tetramethyl ammonium salt. Examples of the pharmaceutically acceptable organic amine addition salts include heterocyclic amine salts such as morpholine and piperidine salts. Examples of the pharmaceutically acceptable amino acid addition salts are salts with lysine, glycine and phenylalanine.

In a preferred embodiment the invention provides compounds according to formula I(a) as follows:

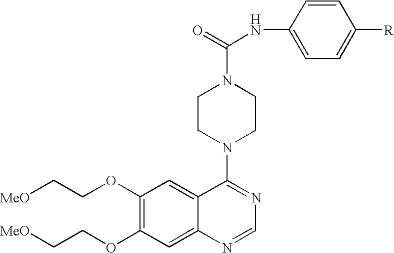

wherein

R is defined as described above for formula I,

and all pharmaceutically acceptable isomers, salts, hydrates, solvates and prodrug derivatives thereof.

In another preferred embodiment the invention provides compounds according to formula I(b) as follows:

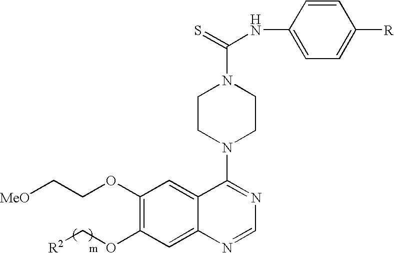

wherein

R, R² and m are each independently defined as described above for formula I,

and all pharmaceutically acceptable isomers, salts, hydrates, solvates and prodrug derivatives thereof.

In another preferred embodiment the invention provides compounds according to formula I(c) as follows:

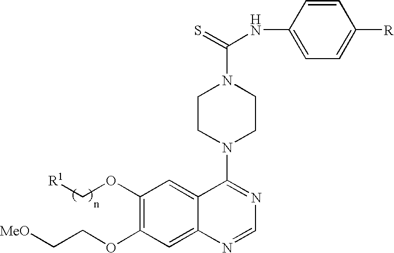

wherein

R, R¹ and n are each independently defined as described above for formula I,

and all pharmaceutically acceptable isomers, salts, hydrates, solvates and prodrug derivatives thereof.

Further, an especially preferred embodiment of the present invention is a compound selected from the group consisting of:

{4-[6,7-bis(2-methoxyethoxy)quinazolin-4-yl]piperazinyl}-N-(4-phenoxyphenyl)carboxamide

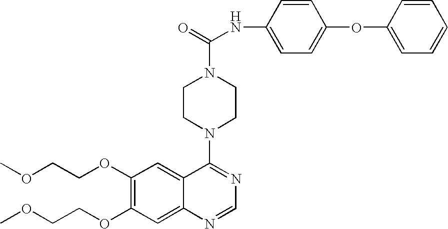

{4-[6,7-bis(2-methoxyethoxy)quinazolin-4-yl]piperazinyl}-N-(4-naphthyloxyphenyl)carboxamide

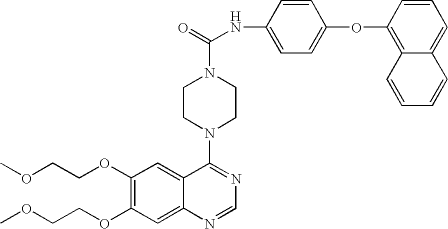

{4-[6,7-bis(2-methoxyethoxy)quinazolin-4-yl]piperazinyl}-N-(4-(5-isoquinolyloxy)phenyl)carboxamide

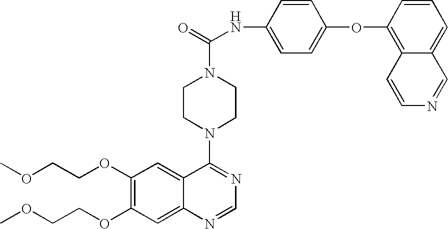

{4-[6,7-bis(2-methoxyethoxy)quinazolin-4-yl]piperazinyl}-N-(4-indol-4-yloxyphenyl)carboxamide

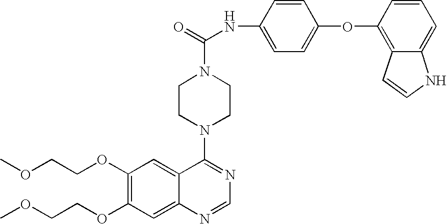

{4-[6-(2-methoxyethoxy)-7-(2-piperidylethoxy)quinazolin-4-yl]piperazinyl}-N-(4-phenoxyphenyl)carboxamide

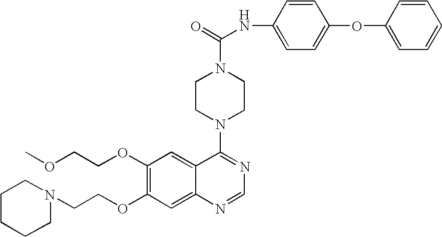

N-(4-cyanophenyl){4-[6-(2-methoxyethoxy)-7-(2-piperidylethoxy)quinazolin-4-yl]piperazinyl}carboxamide

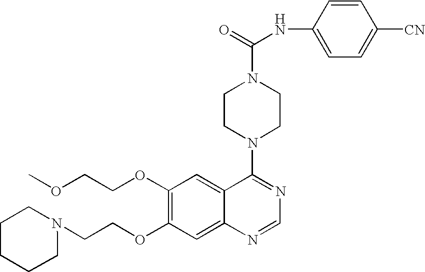

{4-[6-(2-methoxyethoxy)-7-(2-piperidylethoxy)quinazolin-4-yl]piperazinyl}-N-[4-(methylethoxy)phenyl]carboxamide

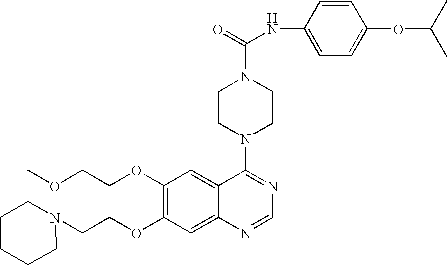

N-(4-cyanophenyl){4-[6-(2-methoxyethoxy)-7-(2-morpholin-4-ylethoxy)quinazolin-4-yl]piperazinyl}carboxamide

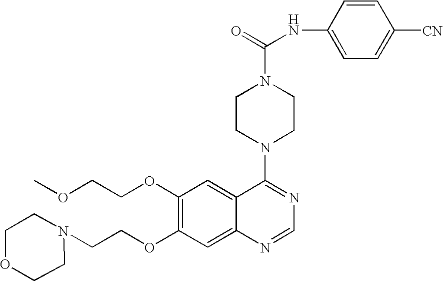

{4-[6,7-bis(2-methoxyethoxy)quinazolin-4-yl]piperazinyl}-N-[4-(3-chloro-4-methylphenoxy)phenyl]carboxamide

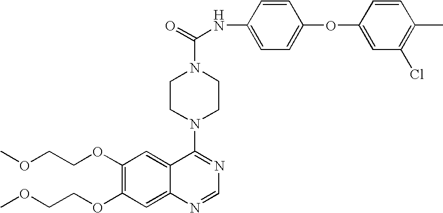

{4-[6,7-bis(2-methoxyethoxy)quinazolin-4-yl]piperazinyl}-N-[4-(4-bromophenoxy)phenyl]carboxamide

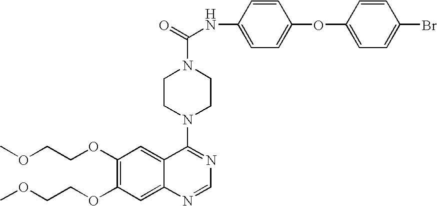

{4-[6,7-bis(2-methoxyethoxy)quinazolin-4-yl]piperazinyl}-N-(4-cyclopropylphenyl)carboxamide

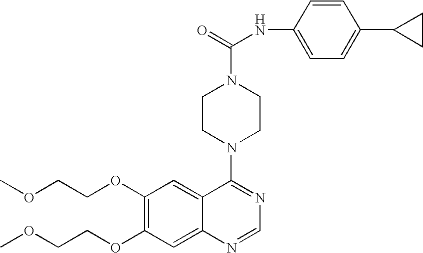

{4-[6,7-bis(2-methoxyethoxy)quinazolin-4-yl]piperazinyl}-N-(4-(5-1,2,3,4-tetrahydroisoquinolyloxy)phenyl)carboxamide

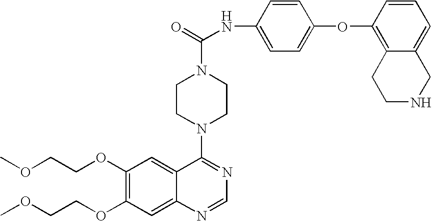

{4-[6,7-bis(2-methoxyethoxy)quinazolin-4-yl]piperazinyl}-N-(4-indolin-4-yloxyphenyl)carboxamide

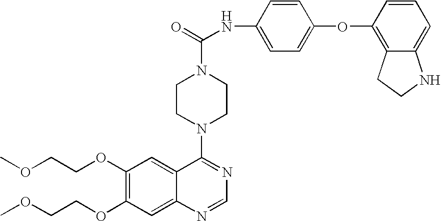

{4-[6-(2-methoxyethoxy)-7-(2-morpholin-4-ylethoxy)quinazolin-4-yl]piperazinyl}-N-[4-(methylethoxy)phenyl]carboxamide

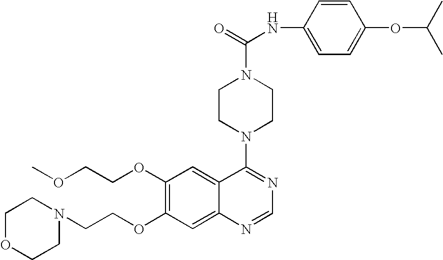

{4-[7-(2-methoxyethoxy)-6-(2-piperidylethoxy)quinazolin-4-yl]piperazinyl}-N-[4-(methylethoxy)phenyl]carboxamide

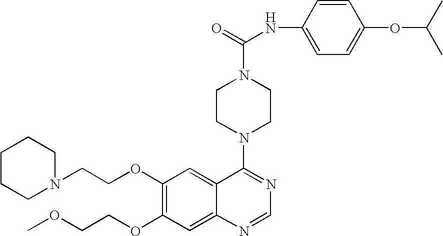

N-(4-cyanophenyl){4-[7-(2-methoxyethoxy)-6-(2-piperidylethoxy)quinazolin-4-yl]piperazinyl}carboxamide

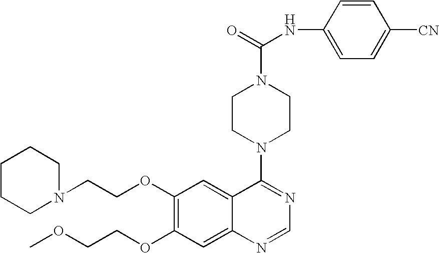

{4-[6-(2-methoxyethoxy)-7-(2-pyrrolidinylethoxy)quinazolin-4-yl]piperazinyl}-N-[4-(methylethoxy)phenyl]carboxamide

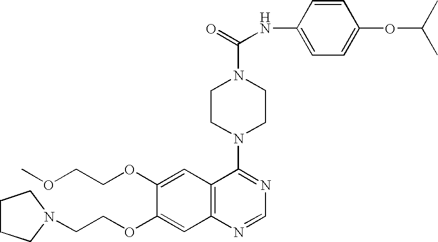

N-(4-cyanophenyl){4-[6-(2-methoxyethoxy)-7-(2-pyrrolidinylethoxy)quinazolin-4-yl]piperazinyl}carboxamide

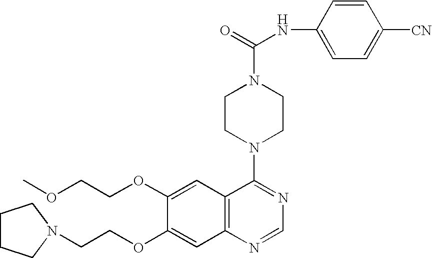

and all pharmaceutically acceptable isomers, salts, hydrates, solvates and prodrug derivatives thereof.

The compounds may be prepared using methods and procedures as generally described in WO 98/14431 published Sep. 12, 1998, which is incorporated herein by reference. Starting materials may be made or obtained as described therein as well. Leaving groups such as halogen, lower alkoxy, lower alkylthio, lower alkylenesulfonyloxy, arylsulfonyloxy, etc, may be utilized when necessary except for the reaction point, followed by deprotection. Suitable amino protective groups are, for example, those described in T. W. Greene, Protective Groups in Organic Synthesis, John Wiley & Sons Inc. (1981), etc., such as ethoxycarbonyl, t-butoxycarbonyl, acetyl and benzyl. The protective groups can be introduced and eliminated according to conventional methods used in organic synthetic chemistry [e.g., T. W. Greene, Protective Groups in Organic Synthesis, John Wiley & Sons Inc. (1981)].

In such processes, if the defined groups change under the conditions of the working method or are not appropriate for carrying out the method, the desired compound can be obtained by using the methods for introducing and eliminating protective groups which are conventionally used in organic synthetic chemistry [e.g., T. W. Greene, Protective Groups in Organic Synthesis, John Wiley & Sons Inc. (1981)], etc. Conversion of functional groups contained in the substituents can be carried out by known methods [e.g., R. C. Larock, Comprehensive Organic Transformations (1989)] in addition to the above-described processes, and some of the active compounds of formula I may be utilized as intermediates for further synthesizing novel derivatives according to formula I.

The intermediates and the desired compounds in the processes described above can be isolated and purified by purification methods conventionally used in organic synthetic chemistry, for example, neutralization, filtration, extraction, washing, drying, concentration, recrystallization, and various kinds of chromatography. The intermediates may be subjected to the subsequent reaction without purification.

There may be tautomers for some formula I, and the present invention covers all possible isomers including tautomers and mixtures thereof. Where chiral carbons lend themselves to two different enantiomers, both enantiomers are contemplated as well as procedures for separating the two enantiomers.

In the case where a salt of a compound of formula I is desired and the compound is produced in the form of the desired salt, it can be subjected to purification as such. In the case where a compound of formula I is produced in the free state and its salt is desired, the compound of formula I is dissolved or suspended in a suitable organic solvent, followed by addition of an acid or a base to form a salt.

The following non-limiting reaction Schemes I and II illustrate preferred embodiments of the invention with respect to making compounds according to the invention.

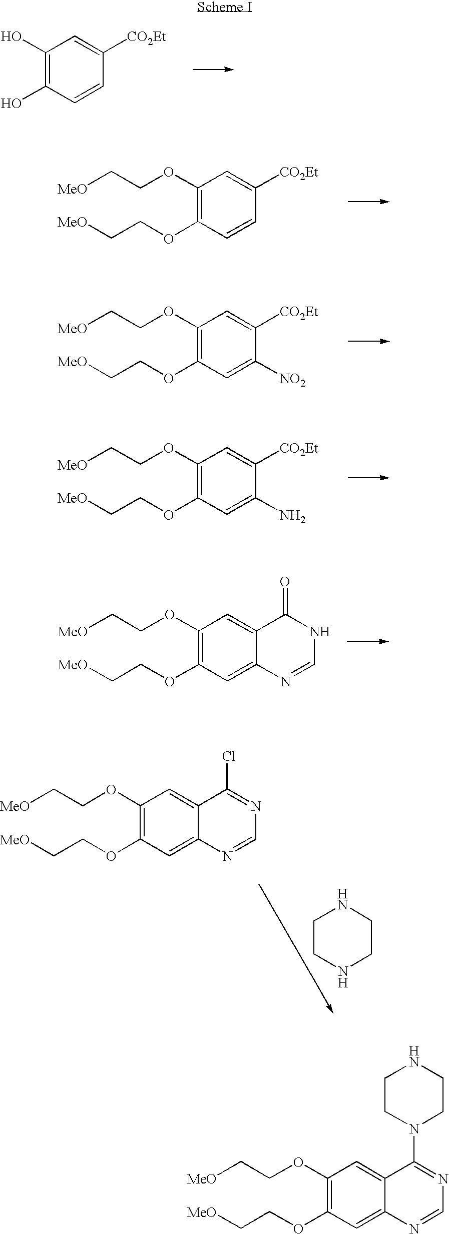

This synthesis of 6,7-bis(2-methoxyethoxy)-4-piperazinylquinazoline compound, provides an intermediate that can be utilized in the synthesis of various compounds (the scheme can be adapted to produce bicyclic position isomers) as described above for formula I. The ethyl 3,4-dihydroxybenzoate is alkylated with 2-bromoethylmethyl ether, followed by nitration with potassium nitrate at room temperature. The nitro functionality is reduced under hydrogenation condition, followed by cyclization with formamide at elevated temperature, preferably in the range 100 to 200° C. to afford quinazolinone. The synthesis of 4-Cl-quinazoline is effected by treating 4-quinazolinone with halogenating reagents such as thionyl chloride, oxalyl chloride or phosphorous oxychloride in presence of solvent such as toluene, or carbon tetrachloride. The key intermediate is obtained by treating 4-Cl-quinazoline with piperazine in an appropriate solvent, such as isopropanol, acetonitrile, or THF at room or reflux temperature for 1-6 h in presence of base triethylamine or pyridine.

Scheme 2 (below) provides the synthesis of various substituted urea intermediates from the intermediate obtained above in Scheme 1, or by other procedures. The treatment of intermediate formed in Scheme 1 with various isocyanates afforded the final urea compounds. In cases where the isocyanates are not commercially available, the piperazine intermediate may be treated with phosgene to give a carbamoyl chloride intermediate followed by reaction with various substituted anilines. The piperazine intermediate can also be treated with p-nitrophenyl chloroformate to afford a nitrophenyl carbamate intermediate that can be treated with various anilines to afford the desired ureas. If the urea compound has a terminal NH₂ group (or one or more of the hydrogen atoms on this amino group is replaced by a displaceable substituent), then this compound may be utilized an intermediate compound with which to produce a urea compound terminated with a —NH-phenyl-R¹ groups. Alternatively, if a different R¹ group is desired on the phenyl group, a replaceable para position leaving group phenyl substituent may be displaced after coupling to provide the particular R¹ substituent as described for formula I, above.

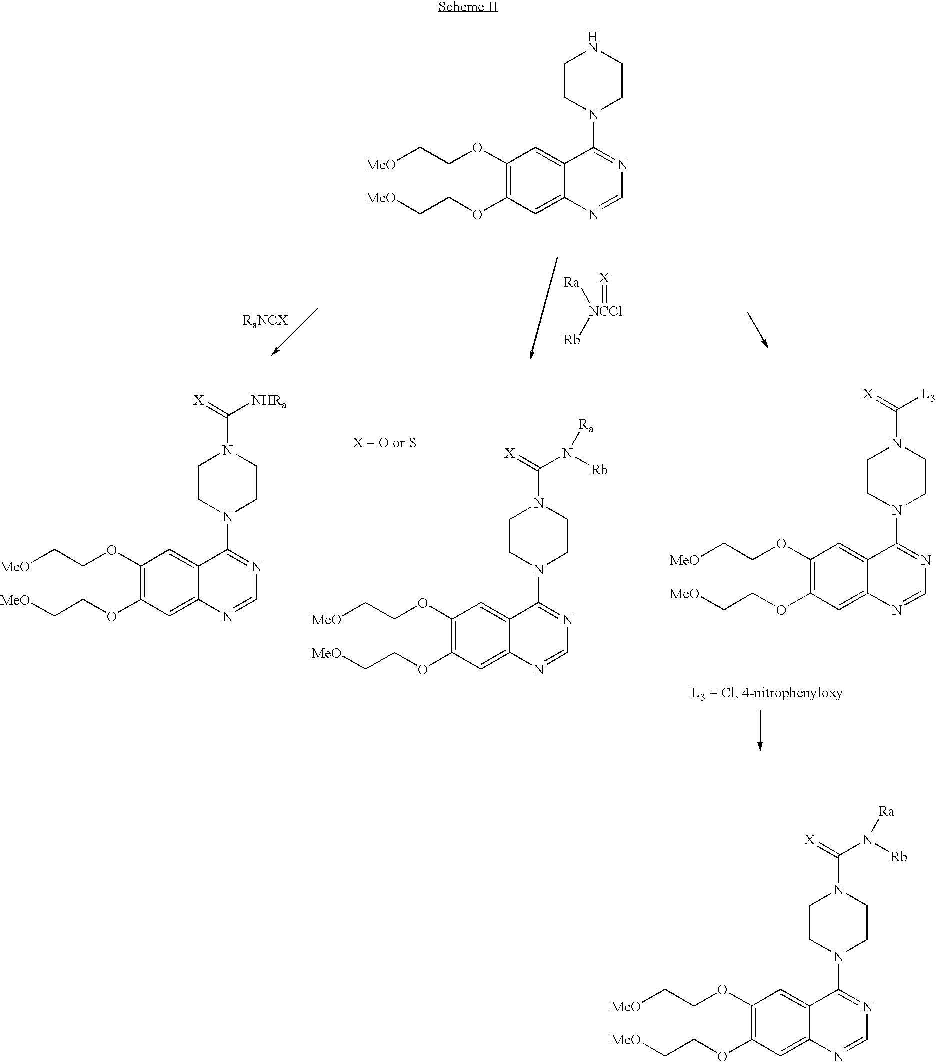

Such procedures for producing the claimed compounds are merely an illustration of a preferred aspect of the invention. Other procedures and adaptations will be apparent to one of ordinary skill in the art upon views these reaction schemes and the structures of the compounds according to the invention. Such procedures are deemed to be within the scope of the present invention.

Also, the compounds of formula I and pharmaceutically acceptable salts thereof may exist in the form of adducts with water (hydrates) or various solvents, which are also within the scope of the present invention.

The following non-limiting examples are provided to better illustrate the present invention.

### EXAMPLE 1

The intermediate 6,7-bis(2-methoxyethoxy)-4-piperazinyquinazoline

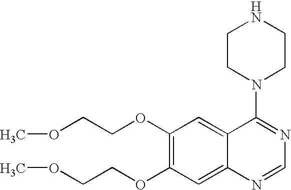

was prepared using the procedures as generally described in reaction Schemes I as follows:

Step A: To the acetone solution (10 mL) of ethyl 3,4-dihydroxybenzoate (0.910 g, 5 mmol), K₂CO₃ (1.52 g, 11 mmol), nBu₄NI (20 mg) was added 2-bromoethylmethyl ether (1.17 mL, 12.5 mmol). The mixture was stirred overnight at 100° C. under argon. After cooling, to this added ether and the precipitate was filtered, the filtrate was evaporated to afford the desired product as white solid (0.850 g, 60%).

Step B: To the acetic acid (4 mL) solution of ethyl-3,4-bis(2-methoxyethoxy)benzoate (0.710 g, 2.4 mmol) at −10° C. was slowly added nitric acid ( 0.350 mL). After stirring at this temperature for 1 h, added KNO₃ (0.240 g, 2.4 mmol) and H₂SO₄ (0.180 mL) followed by stirring at room temperature overnight. The reaction was poured in ice and extracted with EtOAc, washed with brine, dried, filtered and evaporated to give brown oil (0.800 g, quantitative). MS(ES) 344 (M+H)⁺

Step C: To the EtOH (10 mL) solution of nitro material from Step B (0.686 g, 2 mmol) added PtO₂ (40 mg, 0.16 mmol) and then the mixture was shaken on Parr hydrogenation apparatus at 45 psi of H₂ for overnight. Filtration of the catalyst through celite and evaporation of the solvent under vacuum gave the desired amino ester (0.600 g). MS(ES) 314 (M+H)

Step D: The amino product (1 g, 3.21 mmol) from Step C was dissolved in formamide (4 mL) to this added ammonium formate (0.298 g, 4.71 mmol) and the reaction mixture was heated at 150° C. for 4 h. After cooling to room temperature, the solution was diluted with water and extracted with EtOAc. The EtOAc extracts were washed with brine, dried, filtered and evaporated to give beige-colored solid (0.600 g, 62%).

Step E: A mixture of 6,7-bis(2-methoxyethoxy)-4-quinazolinone (0.969 g, 3.3 mmol) and POCl₃ (4.5 mL) was heated to reflux under argon for 6 h. After cooling to 0° C., added dropwise conc. NH₄OH, a solid precipitated which was collected by filtration. The solid collected was washed with water and dried under high vacuum to afford desired 4-chloroquinazoline.

Step F: To the isopropanol solution (5 mL) of 6,7-bis(2-methoxyethoxy)-4-Cl-quinazoline (0.156 g, 0.5 mmol, from Step E) added piperazine (0.300 g, 3.5 mmol) and the reaction mixture was refluxed for 3 h. The solvent was evaporated and the residue dissolved in EtOAc and washed with brine, dried, filtered and evaporated to give desired key intermediate 6,7-bis(2-methoxyethoxy)-4-piperazinyquinazoline as a white solid (0.1 50 g). MS(ES) 363(M+H)

### EXAMPLE 2

{4-[6,7-bis(2-methoxyethoxy)quinazolini-4-yl]piperazinyl}-N-(4-cyanophenyl)piperazinyl carboxamide was prepared using the intermediate obtained in Example 1 and the procedures as generally described in reaction Scheme II as follows:

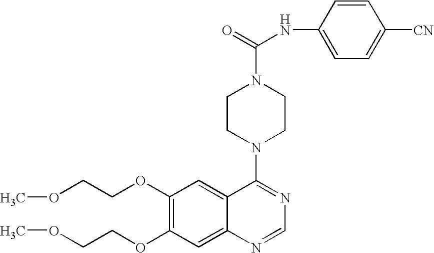

To DMF solution (1 mL) of 6,7-bis(2-methoxyethoxy)-4-piperazinyquinazoline from Step F Example 1 (0.100 g, 0.27 mmol) was added DMF solution (1 mL) of 4-cyanophenylisocyanate (75 mg, 0.45 mmol) and the reaction was stirred at room temperature overnight. The solvent was evaporated and residue purified by RP-HPLC to afford desired product {4-[6,7-bis(2-methoxyethoxy)quinazolin-4-yl]piperazinyl}-N-(4-cyanophenyl)piperazinyl carboxamide as a white solid (0.100, 73%). MS(ES) 507(M+H)⁺

### EXAMPLES 3

{4-[6,7-bis(2-methoxyethoxy)quinazolin-4-yl]piperazinyl}-N-(4-phenoxyphenyl)carboxamide was prepared using the following procedure:

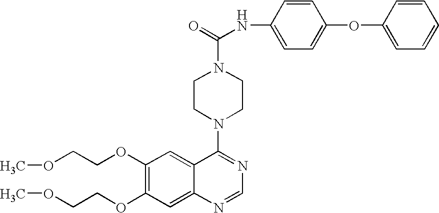

To the acetonitrile solution (1 mL) of triphosgene (26.4 mg, 0.09 mmol) at −5° C. added a solution of 4-phenoxyaniline (91.6 mg, 0.26 mmol, prepared as described in Example 3, Step B) and Et₃N (73.2 □L) in acetonitrile (1 mL) over a period of 5 min. To this was added a acetonitrile solution (1 mL) of 6,7-bis(2-methoxyethoxy)-4-piperazinyquinazoline (125 mg, 0.09 mmol, Example 1, Step F) and Et₃N (73.2 □L). The reaction slowly warmed up to room temperature and stirred for 3 h. The solvent was evaporated and the residue purified by RP-HPLC (reverse-phase high performance liquid chromatography) to afford desired {4-[6,7-bis(2-methoxyethoxy)quinazolin-4-yl]piperazinyl}-N-(4-phenoxyphenyl)carboxamide as a flaky shiny white solid (132 mg, 75%). MS(ES) 574 (M+H)

The starting material 4-phenoxyaniline was synthesized as follows:

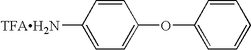

Step A: To the anhydrous DMSO solution (7 mL) of phenol (0.44 g, 4.7 mmol) added NaH (0.24 g, 6.05 mmol) and stirred for 45min at room temperature. To this added 1-fluoro-4-nitrobenzene(0.66 g, 4.65 mmol) and this was heated at 90° C. for overnight. After cooling diluted with EtOAc and washed with brine, dried, filtered, evaporated to afford crude residue. This was purified by silica gel chromatography (10%EtOAc/hexane) to give desired product as bright yellow oil (0.91 g, 91%). MS(ES) 216 (M+H)

Step B: The nitro compound from Step A (0.885 g, 4.12 mmol) was dissolved in EtOH/EtOAc (10 mL, 1:1), to this added 10%Pd/C (0.100 g) and TFA (0.317 mL, 4.12 mmol). The reaction mixture was stirred under 1 atm H₂ for overnight, filtered through celite. The filtrate was concentrated in vacuo to afford 4-phenoxyaniline as a pink solid (1.15 g, 93.5%). MS(ES) 186 (M+H)⁺

### EXAMPLES 4

tert-butyl-4-[6-(2-methoxyethoxy)-7-(2-piperidylethoxy)quinazolin-4-yl]piperazinylcarboxylate:

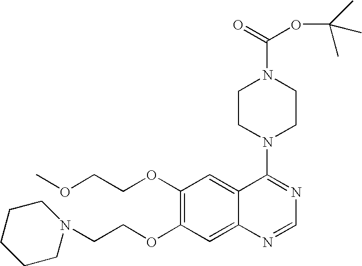

was prepared using the following procedure:

Step A: To the DMF solution (12 mL) of Ethyl-3-hydroxy-4-fluorobenzoate (0.80 g, 4.35 mmol) added K₂CO₃ (2.10 g, 15.22 mmol) followed by 2-bromoethyl methylether (0.45 mL, 4.76 mmol). The reaction mixture was heated at 70° C. for overnight, after cooling added water and extracted the product with EtOAc. The EtOAc layer was dried, filtered and evaporated to afford desired product as pale yellow oil (1.04 g, 90%).

Step B: To a DMF solution (10 mL) of 2-piperidineethanol (2.86 mL, 21.6 mmol) added NaH(0.83 g, 20.9 mmol) at 0° C. and the mixture was stirred for 1 h. To this added DMF solution (3 mL) of ester (0.725 g, 3.15 mmol, from Step A, Example 4) and heated the reaction at 90° C. overnight. The solvent was evaporated and the residue purified by RP-HPLC to afford the desired ethyl 3-(2-methoxyethoxy)-4-(2-piperidylethoxy)benzoate as a off-white solid. MS(ES) 352(M+H)

Step C: To the acetic acid (3 mL) solution of ethyl 3-(2-methoxyethoxy)-4-(2-piperidylethoxy)benzoate (0.197 g, 0.56 mmol) at −10° C. was slowly added nitric acid (0.200 mL). After stirring at this temperature for 1 h, added KNO₃ (0.056 g, 0.56 mmol) and H₂SO₄ (0.200 mL) slowly warmed up to room temperature and stirred for 1 h. The reaction was poured in ice, basified with NH4OH, and extracted with EtOAc. The EtOAc layer was separated, dried, filtered and evaporated to give white solid (0.200 g, 95%). MS(ES) 397 (M+H)⁺

Step D: To the EtOH (10 mL) solution of nitro material from Step C (0.200 g, 0.5 mmol) added 10%Pd/C and then the mixture was stirred under 1 atm of H₂ for overnight. Filtration of the catalyst through celite and evaporation of the solvent under vacuum gave the desired amino ester (0.162 g). MS(ES) 367 (M+H)

Step E: The amino product (0.160 g, 0.437 mmol) from Step D was dissolved in formamide (0.278 mL, 7 mmol)) to this added ammonium formate (0.256 g, 4.36 mmol) and the reaction mixture was heated at 135° C. for 4 h. After cooling to room temperature, the solution was diluted with water and extracted with EtOAc. The EtOAc extracts were washed with brine, dried, filtered and evaporated to give off-white solid (0.198 g, 96%). MS(ES) 370 (M+Na).

Step F: To 4-quinazolinone (0.195 g, 0.423 mmol, From Step E) added SOCl₂ (3 mL), DMF (0.200 mL) and the mixture was heated at 90° C. for 1 h. After cooling evaporated the solvent and azetroped several times with toluene to afford the desired 4-Cl-quinazoline as tan crystalline solid (0.197 g). ES(MS) 366 (M+H, with 1 Cl).

Step G: To the isopropanol solution (8 mL) of the material from Step F (0.190 g, 0.423 mmol) added Boc-piperazine (0.197 g, 1.06 mmol) followed by Et₃N (0.288 mL, 2.11 mmol). The reaction mixture was heated to reflux overnight. The solvent was evaporated and the residue purified by RP-HPLC to afford desired product tert-butyl-4-[6-(2-methoxyethoxy)-7-(2-piperidylethoxy)quinazolin-4-yl]piperazinylcarboxylate (0.173 g, 62%). MS(ES) 516 (M+H).

### EXAMPLE 5

N-(4-cyanophenyl)piperazinyl{4-[6-(2-methoxyethoxy)-7-(2-piperidylethoxy)quinazolin-4-yl]piperazinyl}carboxamide was prepared using the intermediate obtained in Example 2 and the procedures as generally described in reaction Scheme II as follows:

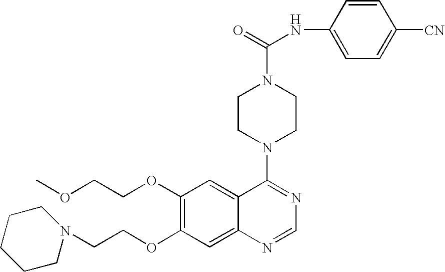

To tert-butyl-4-[6-(2-methoxyethoxy)-7-(2-piperidylethoxy)quinazolin-4-yl]piperazinylcarboxylate (from Example 4, Step G, 0.055 g, 0.107 mmol) added 4N HCl/dioxane (1 mL) and reaction was stirred at rom temperature for 1 h. The solvent was evaporated to afford deboc material, (i.e. material with the Boc protecting group removed), to this added DMF (1 mL), Et₃N (0.200 mmol) followed by DMF solution (1 mL) of 4-cyanophenylisocyanate (20 mg, 0.133 mmol) and the reaction was stirred at RT overnight. The solvent was evaporated and residue purified by RP-HPLC to afford desired product N-(4-cyanophenyl)piperazinyl{4-[6-(2-methoxyethoxy)-7-(2-piperidylethoxy)quinazolin-4-yl]piperazinyl}carboxamide as a white solid (0.053 g, 73%). MS(ES) 560(M+H)⁺

The pharmacological activities of the compounds of the present invention are obtained by following the test example procedures as follows, for example.

Biological Test Assay Type 1

Inhibitory Effect on Compounds on Autophosphorylation of Platelet Derived Growth Factor β-PDGF Receptor

(1) HR5 Phosphorylation Assay

The HR5 cell line is a cell line of CHO cells engineered to overexpress human β-PDGFR, which cell line is available from the ATCC. The expression level of β-PDGFR in HR5 cells is around 5×10⁴ receptor per cell. For the phosphorylation assay according to the invention, HR5 cells were grown to confluency in 96-well microtiter plates under standard tissue culture conditions, followed by serum-starvation for 16 hours. Quiescent cells were incubated at 37° C. without or with increasing concentrations of the test compound (0.01-30 μM) for 30 minutes followed by the addition of 8 nM PDGF BB for 10 minutes. Cells were lysed in 100 mM Tris, pH7.5, 750 mM NaCl, 0.5% Triton X-100, 10 mM sodium pyrophosphate, 50 mM NaF, 10 ug/ml aprotinin, 10 ug/ml leupeptin, 1 mM phenylmethylsulfonyl fluoride, 1 mM sodium vanadate, and the lysate was cleared by centrifugation at 15,000×g for 5 minutes. Clarified lysates were transferred into a second microtiter plate in which the wells were previously coated with 500 ng/well of 1B5B11 anti-βPDGFR mAb, and then incubated for two hours at room temperature. After washing three times with binding buffer (0.3% gelatin, 25 mM Hepes pH 7.5, 100 mM NaCl, 0.01% Tween-20), 250 ng/ml of rabbit polyclonal anti-phosphotyrosine antibody (Transduction Laboratory) was added and plates were incubated at 37° C. for 60 minutes. Subsequently, each well was washed three times with binding buffer and incubated with 1 ug/ml of horse radish peroxidase-conjugated anti-rabbit antibody (Boehinger Mannheim) at 37° C. for 60 minutes. Wells were washed prior to adding ABTS (Sigma), and the rate of substrate formation was monitored at 650 nm. The assay results are reported as IC₅₀ (expressed as the concentration of a compound according to the invention that inhibits the PDGF receptor phosphorylation by 50%) as compared to control cells that are not exposed to a compound according to the invention.

Examples of such IC₅₀ test results in the HR5 assay for compounds according to the invention are set forth below in Table 1.

(2) MG63 Phosphorylation Assay

The MG63 cell line is a human osteosarcoma tumor cell line available from the ATCC. This assay is for measuring endogenous β-PDGFR phosphorylation in MG63 cells. The assay conditions are the same as those described for HR5 cell, except that PDGF-BB stimulation is provided in the presence or absence of 45% human plasma. The MG63 assay results are reported as an IC₅₀ (expressed as the concentration of a compound according to the invention that inhibits the PDGF receptor phosphorylation by 50%) as compared to control cells that are not exposed to a compound according to the invention. Examples of such IC₅₀ test results in the MG63 assay for compounds according to the invention are set forth below in Table 1.

The assay results for Compound Examples 2 and 3 are set forth in Table 1 below.

TABLE 1

| Example | MG63 w/human plasma | HR5 |
|---|---|---|
| Compound | IC₅₀ (μM) | IC₅₀ (μM) |
| Example 2 | 0.615 | 1.34 |
| Example 3 | 0.174 | 0.050 |
| Example 5 | 0.090 | 0.030 |

Biological Test Assay Type 2

Growth Inhibition Against Smooth Muscle Cells

Vascular smooth muscle cells are isolated from a pig aorta by explanation and used for the test. The cells are put into wells of a 96-well plate (8000 cells/well) and cultured in Dulbeccois modified Eagle's medium (DMEM; Nissui Pharmaceutical Co., Ltd.) containing 10% fetal bovine serum (FBS; Hyclone) for 4 days. Then, the cells are further cultured in DMEM containing 0.1% FBS for 3 days, and are synchronized at the cell growth stationary phase.

To each well is added DMEM containing 0.1% FBS and a test sample at a varied concentration, and the cell growth is brought about by PDGF-BB (SIGMA, final concentration: 20 ng/ml). After culturing for 3 days, the cell growth is measured using a cell growth assay kit (Boehringer Mannheim) according to the XTT method [J. Immunol. Methods, 142, 257-265 (1991)], and the cell growth score is calculated by the following equation.

Cell growth score=100×{1−(M−PO)/(P100−PO)} wherein P100=absorbance by XTT reagent when stimulated by PDGF-BB; PO=absorbance by XTT reagent when not stimulated by PDGF-BB, and M=absorbance by XTT reagent after addition of a sample when stimulated by PDGF-BB.

The test result is expressed as the concentration of a test compound which inhibits the cell growth by 50% (IC₅₀).

Biological Test Assay Type 3

Inhibitory effect on Hypertrophy of Vascular Intima

Male SD rats (weight: 375-445 g, Charles River, golden standard) are anesthetized with sodium pentobarbital (50 mg/kg, i.p.), and then the neck of each animal is incised by the median incision, followed by retrograde insertion of a balloon catheter (2F, Edwards Laboratories) into the left external carotid. After the above treatment is repeated seven times, the catheter is pulled out, the left external carotid is ligated, and the wound is sutured. A test compound is suspended in a 0.5% solution of Tween 80 in an aqueous solution of sodium chloride to a concentration of 20 mg/ml in the case of intraperitoneal administration and in a 0.5% solution of methyl cellulose 400 to a concentration of 6 mg/ml in the case of oral administration. The suspension is administered once a day in the case of intraperitoneal administration and once or twice a day in the case of oral administration for a period of 15 days starting on the day before the balloon injury. On the 14th day after the balloon injury, the animal is killed and its left carotid is extirpated. The tissues are fixed with formalin, wrapped in paraffin and sliced, followed by Elastica Wangeeson staining. The area of the cross section of the vascular tissues (intima and media) is measured with an image analyzer (Luzex F, NIRECO) and the intima/media area ratio (I/M) is regarded as the degree of hypertrophy of the vascular intima.

From the results obtained, it is apparent when the hypertrophy of vascular intima is significantly inhibited by administration of the compounds of the present invention.

Biological Test Assay Type 4

Evaluation by the Use of a Rat Adjuvant Arthritis Model

Dead cells of Mycobacterium bacterium (Difco Laboratories Inc.) are disrupted in agate mortar and suspended in liquid paraffin to the final concentration of 6.6 mg/ml, followed by sterilization with high pressure steam. Then, 100 ml of the suspension is subcutaneously injected into the right hind foot pad of each animal of groups of female 8-weeks-old Lewis rats (Charles River Japan) (6 animals/group) to induce adjuvant arthritis. A test compound i suspended in a 0.5% solution of methyl cellulose to the final concentration of 3 mg/ml, and from just before the induction of arthritis, the suspension is orally administered in an amount of 100 ml/100 g of the body weight once a day, 5 days a week. To a control group is administered a 0.5% solution of methyl cellulose. A normal group is given no adjuvant treatment or test compound administration. The administration of the test compound is continued till the 18th day after the adjuvant treatment. On the 17th day, the number of leukocytes in peripheral blood are counted, and on the 18th day, all the blood is collected, followed by dissection.

The change in body weight with the passage of time, the change of edema in hind foot with the passage of time, the weight of spleen and thymus, the number of leukocytes in peripheral blood, the hydroxyproline content of urine, the glucosaminoglycan content of urine, the SH concentration in serum, the concentration of nitrogen monoxide in serum and the concentration of mucoprotein in serum are measured and evaluated. The volume of each of both hind feet are measured using a rat's hind foot edema measurement device (TK-101, Unicom). The number of leukocytes in peripheral blood are counted using an automatic multichannel blood cell counter (Sysmex K-2000, Toa Iyo Denshi Co., Ltd.). The hydroxyproline content of urine is measured according to the method described in Ikeda, et al., Annual Report of Tokyo Metropolitan Research Laboratories P. H.,, 36, 277 (1985), and the glucosaminoglycan content is measured according to the method described in Moriyama, et al., Hinyo Kiyo, 40, 565 (1994) and Klompmakers, et al., Analytical Biochemistry, 153, 80 (1986). The SH concentration in serum is measured according to the method described in Miesel, et al., Inflammation, 17, 595 (1993), and the concentration of nitrogen monoxide is measured according to the method of Tracey, et al., Journal of Pharmacology & Experimental Therapeutics, 272, 1011 (1995). The concentration of mucoprotein is measured using Aspro GP Kit (Otsuka Pharmaceutical Co., Ltd.). The percentage inhibition for each indication is calculated according to the following equation.

% Inhibition={(Control group−Compound-administered group)/(Control group−Normal group)}×100.

From the results obtain from such assays, it is apparent when the compound according to the invention inhibits the occurrence of adjuvant arthritis.

Biological Test Assay Type 5

Activity On a Mesangial Proliferative Glomerulonephritis Model

Anti-rat Thy-1.1 monoclonal antibody OX-7 (Sedaren) is administered to male Wister-Kyoto rats (Charles River Japan, 160 g, 6 animals/group) in an amount of 1.0 mg/kg by intravenous administration through the tail vein. A test compound is suspended in a 0.5% solution of methylcellulose and the resulting suspension is administered to each of the rats twice a day for a period of 7 days starting on the day before the administration of OX-7. On the 7th day after the OX-7 administration, when mesangial cell growth and extracellular matrix hypertrophy become prominent, the left kidney of each rat is extirpated, fixed with 20% buffered formalin for 6 hours and wrapped in paraffin, followed by slicing. The obtained pieces are subjected to immune tissue staining using antibody PC10(DAKO) against an intranuclear antigen of proliferative cells. After comparative staining with Methyl Green staining solution using diaminobenzidine as a color developer, the paraffin pieces are enclosed. Half of the glomeruli in a kidney piece are observed and the number of the cells in one glomerulus which are positive to the intranuclear antigen of proliferative cells are calculated. The test for the significance of difference is carried out by the Wilcoxon test.

From such results, it is apparent when the compounds according to the present invention show alleviating activity on mesangial proliferative glomerulonephritis.

The compounds of formula (I) and pharmaceutically acceptable salts thereof can be administered as such, but it is usually preferred to administer them in the form of pharmaceutical compositions, which are used for animals and human beings. It is preferred to employ the administration route which is the most effective for the treatment. For example, administration is made orally or non-orally by intrarectal, intraoral, subcutaneous, intramuscular or intravenous administration.

Examples of the forms for administration are capsules, tablets, granules, powders, syrups, emulsions, suppositories and injections.

Liquid compositions such as emulsions and syrups which are appropriate for oral administration can be prepared using water, sugars such as sucrose, sorbitol and fructose, glycols such as polyethylene glycol and propylene glycol, oils such as sesame oil, olive oil and soybean oil, preservatives such as benzoates, flavors such as strawberry flavor and peppermint, etc.

Capsules, tablets, powders and granules can be prepared using excipients such as lactose, glucose, sucrose and mannitol, disintegrating agents such as starch and sodium alginate, lubricants such as magnesium stearate and talc, binders such as polyvinyl alcohol, hydroxypropyl cellulose and gelatin, surfactants such as fatty acid esters, plasticizers such as glycerin, etc.

Compositions suitable for non-oral administration preferably comprise a sterilized aqueous preparation containing an active compound which is isotonic to the recipient's blood. For example, injections are prepared using a carrier which comprises a salt solution, a glucose solution, or a mixture of a salt solution and a glucose solution.

Compositions for topical application are prepared by dissolving or suspending an active compound in one or more kinds of solvents such as mineral oil, petroleum and polyhydric alcohol, or other bases used for topical drugs.

Compositions for intestinal administration are prepared using ordinary carriers such as cacao fat, hydrogenated fat and hydrogenated fat carboxylic acid, and are provided as suppositories.

The compositions for non-oral administration may additionally be formulated to contain one or more kinds of additives selected from glycols, oils, flavors, preservatives (including antioxidants), excipients, disintegrating agents, lubricants, binders, surfactants and plasticizers which are used for the preparation of compositions for oral administration. The effective dose and the administration schedule for each of the compounds of formula (I) or a pharmaceutically acceptable salt thereof will vary depending on the administration route, the patient's age and body weight, and the type or degree of the diseases to be treated. However, it is generally appropriate to administer a compound of formula (I) or a pharmaceutically acceptable salt thereof in a dose of 0.01-1000 mg/adult/day, preferably 5-500 mg/adult/day, in one to several parts.

All the compounds of the present invention can be immediately applied to the treatment of kinase-dependent diseases of mammals as kinase inhibitors, specifically, those relating to tyrosine kinase. Specifically preferred are the compounds which have IC50 within the range of 10 nM-10 μM. Even more preferred are compounds which have IC50 within the range of 10 μM to −1 μM. Most preferred are compounds which have an IC50 value which is smaller than 1 μM.

Specific compounds of the present invention which have an activity to specifically inhibit one of the three types of protein linase (for example, kinase which phosphorylates tyrosine, kinase which phosphorylates tyrosine and threonine, and kinase which phosphorylates threonine) can be selected. Tyrosine kinase-dependent diseases include hyperproliferative malfunction which is caused or maintained by abnormal tyrosine kinase activity. Examples thereof include psoriasis, pulmonary fibrosis, glomerulonephritis, cancer, atherosclerosis and anti-angiopoiesis (for example, tumor growth and diabetic retinopathy). Current knowledge of the relationship between other classes of kinase and specific diseases is insufficient. However, compounds having specific PTK-inhibiting activity have a useful treatment effect. Other classes of kinase have also been recognized in the same manner. Quercetin, genistein and staurosporin, which are all PTK-inhibitors, inhibit many kinds of protein kinase in addition to tyrosine kinase. However, as a result of their lack of the specificity, their cytotoxicity is high. Therefore, a PTK-inhibitor (or an inhibitor of other classes of kinase) which is apt to bring about undesirable side effects because of the lack of selectivity can be identified by the use of an ordinary test to measure cytotoxicity.

The present invention provides nitrogen-containing heterocyclic compounds and pharmaceutically acceptable salts thereof which inhibit phosphorylation of PDGF receptor to hinder abnormal cell growth and cell wandering and thus are useful for the prevention or treatment of cell-proliferative diseases such as arteriosclerosis, vascular reobstruction, cancer and glomerulosclerosis.

Although the present invention has been described in some detail by way of illustration for purposes of clarity of understanding, it will be apparent to those of ordinary skill in the art that various modifications and equivalents can be made without departing from the spirit and scope of the invention. It should be understood that the foregoing discussion and examples merely present a detailed description of certain preferred embodiments. All the patents, journal articles and other documents discussed or cited above are herein incorporated by reference in their entirety.

What is claimed is:

1. A nitrogen-containing heterocyclic compound of the formula: 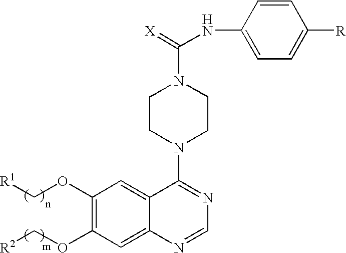
wherein
m and n are each independently from 2 to 8,
X is O or S; and
R is a member selected from the group consisting of: 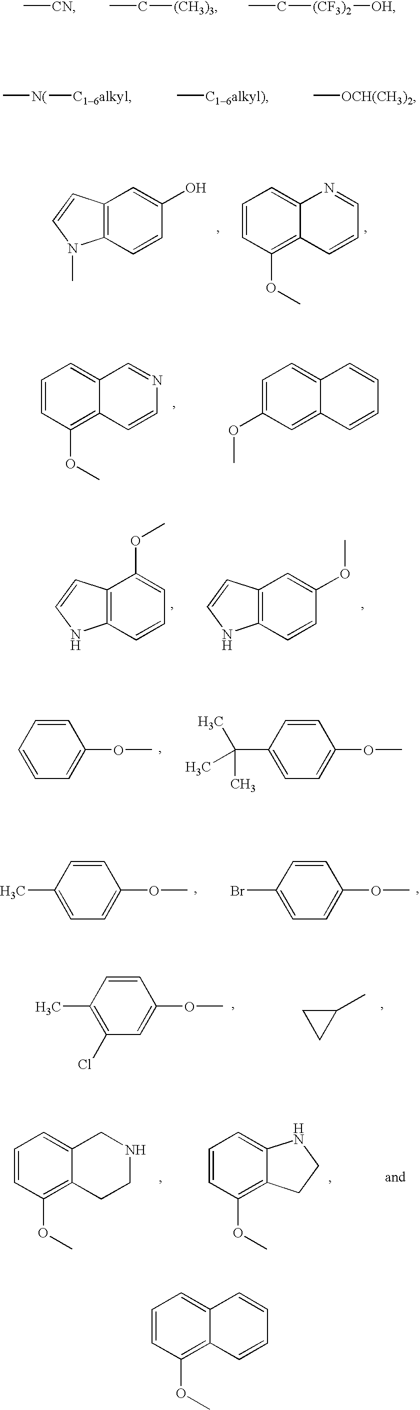
R¹ and R² are each independently a member selected from the group consisting of —OH, —O—CH₃, 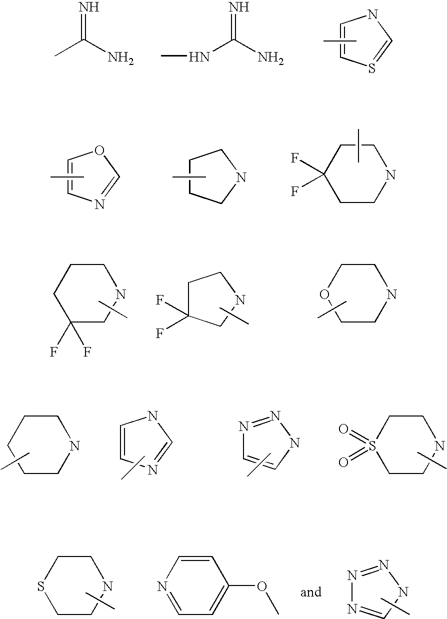
and all pharmaceutically acceptable isomers, salts, hydrates, solvates and prodrug derivatives thereof.

2. The compound of claim 1, having formula I(a) as follows: 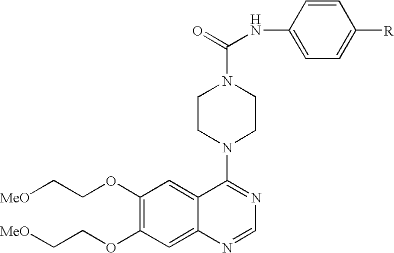
wherein
R is defined as in claim 1,
and all pharmaceutically acceptable isomers, salts, hydrates, solvates and prodrug derivatives thereof.

3. The compound of claim 1, having formula I(b) as follows: 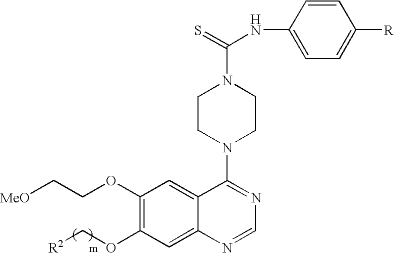
wherein
R, R² and m are each independently defined as described in claim 1 above,
and all pharmaceutically acceptable isomers, salts, hydrates, solvates and prodrug derivatives thereof.

4. The compound of claim 3, wherein m is 2 or 3.
and all pharmaceutically acceptable isomers, salts, hydrates, solvates and prodrug derivatives thereof.

5. The compound of claim 1, having formula I(c) as follows: 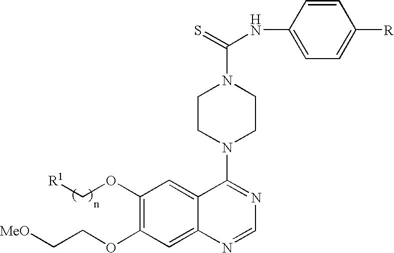
wherein
R, R¹ and n are each independently defined as described above in claim 1,
and all pharmaceutically acceptable isomers, salts, hydrates, solvates and prodrug derivatives thereof.

6. The compound of claim 5, wherein n is 2 or 3,
and all pharmaceutically acceptable isomers, salts, hydrates, solvates and prodrug derivatives thereof.

7. A compound according to claim 1, selected from the group consisting of:
{4-[6,7-bis(2-methoxyethoxy)quinazolin-4-yl]piperazinyl}-N-(4-phenoxyphenyl) carboxamide 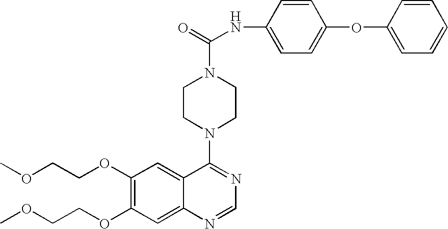
{4-[6,7-bis(2-methoxyethoxy)quinazolin-4-yl]piperazinyl}-N-(4-naphthyloxyphenyl) carboxamide 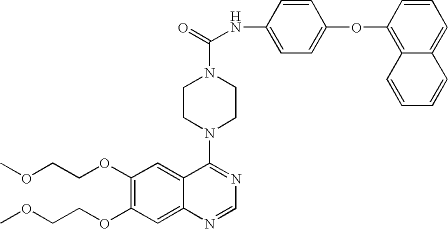
{4-[6,7-bis(2-methoxyethoxy)quinazolin-4-yl]piperazinyl}-N-(4-(5-isoquinolyloxy)phenyl) carboxamide 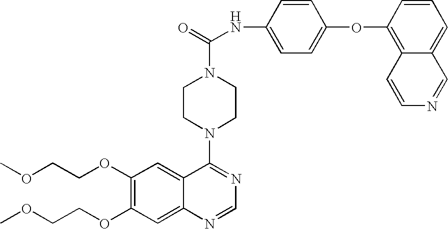
{4-[6,7-bis(2-methoxyethoxy)quinazolin-4-yl]piperazinyl}-N-(4-indol-4-yloxyphenyl) carboxamide 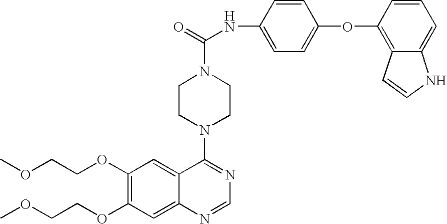
{4-[6,7-bis(2-methoxyethoxy)quinazolin-4-yl]piperazinyl}-N-[4-(3-chloro-4-methylphenoxy)phenyl]carboxamide 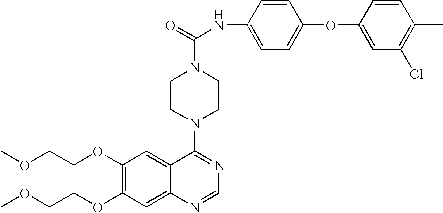
{4-[6,7-bis(2-methoxyethoxy)quinazolin-4-yl]piperazinyl}-N-[4-(4-bromophenoxy)phenyl]carboxamide 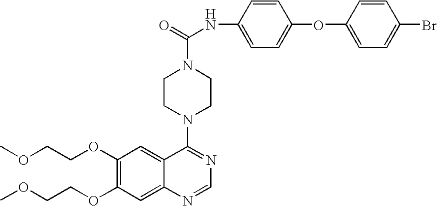
{4-[6,7-bis(2-methoxyethoxy)quinazolin-4-yl]piperazinyl}-N-(4-cyclopropylphenyl) carboxamide 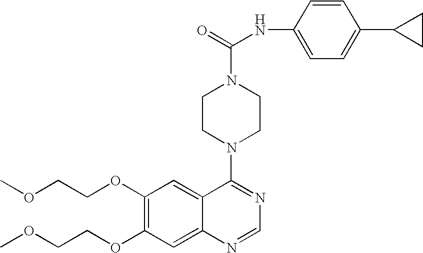
{4-[6,7-bis(2-methoxyethoxy)quinazolin-4-yl]piperazinyl}-N-(4-(5-1,2,3,4-tetrahydroisoquinolyloxy)phenyl)carboxamide 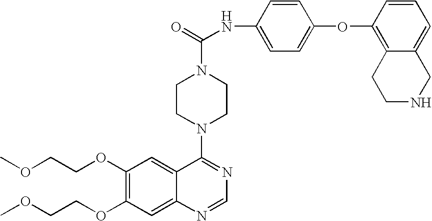
{4-[6,7-bis(2-methoxyethoxy)quinazolin-4-yl]piperazinyl}-N-(4-indolin-4-yloxyphenyl) carboxamide 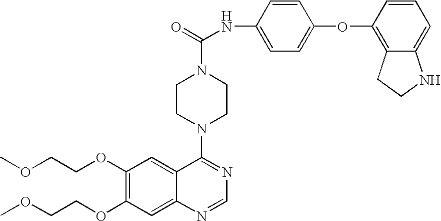
{4-[6-(2-methoxyethoxy)-7-(2-piperidylethoxy)quinazolin-4-yl]piperazinyl}-N-(4-phenoxyphenyl)carboxamide 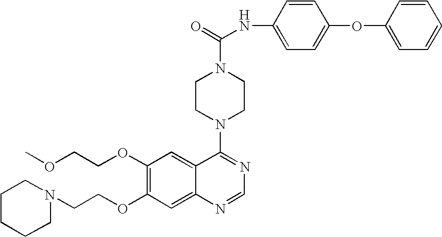
N-(4-cyanophenyl){4-[6-(2-methoxyethoxy)-7-(2-piperidylethoxy)quinazolin-4-yl]piperazinyl}carboxamide 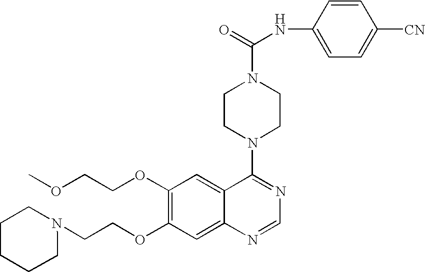
{4-[6-(2-methoxyethoxy)-7-(2-piperidylethoxy)quinazolin-4-yl]piperazinyl}-N-[4-(methylethoxy)phenyl]carboxamide 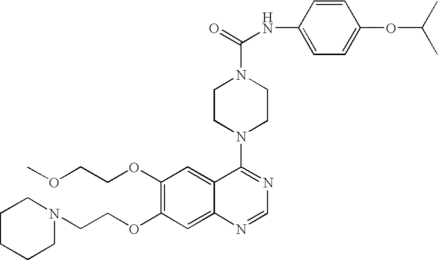
N-(4-cyanophenyl){4-[6-(2-methoxyethoxy)-7-(2-morpholin-4-ylethoxy)quinazolin-4-yl]piperazinyl}carboxamide 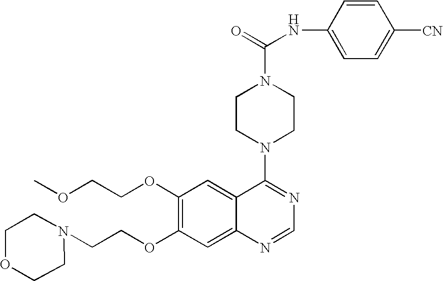
{4-[6-(2-methoxyethoxy)-7-(2-morpholin-4-ylethoxy)quinazolin-4-yl]piperazinyl}-N-[4-(methylethoxy)phenyl]carboxamide 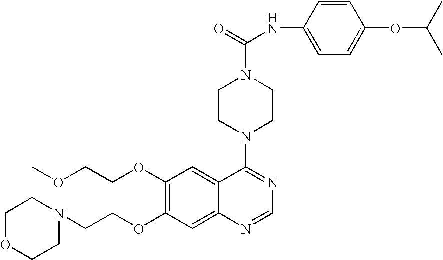
{4-[7-(2-methoxyethoxy)-6-(2-piperidylethoxy)quinazolin-4-yl]piperazinyl}-N-[4-(methylethoxy)phenyl]carboxamide 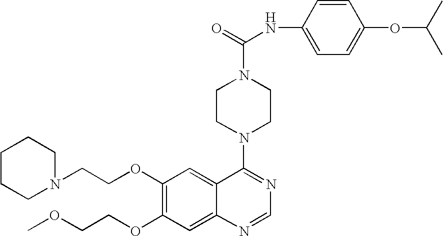
N-(4-cyanophenyl){4-[7-(2-methoxyethoxy)-6-(2-piperidylethoxy)quinazolin-4-yl]piperazinyl}carboxamide 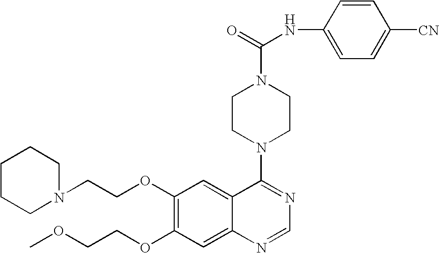
{4-[6-(2-methoxyethoxy)-7-(2-pyrrolidinylethoxy)quinazolin-4-yl]piperazinyl}-N-[4-(methylethoxy)phenyl]carboxamide 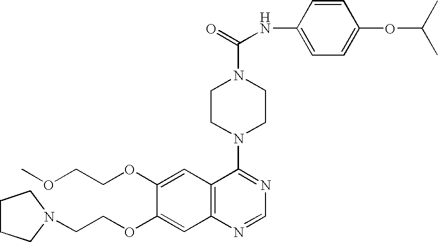
N-(4-cyanophenyl){4-[6-(2-methoxyethoxy)-7-(2-pyrrolidinylethoxy)quinazolin-4-yl]piperazinyl}carboxamide 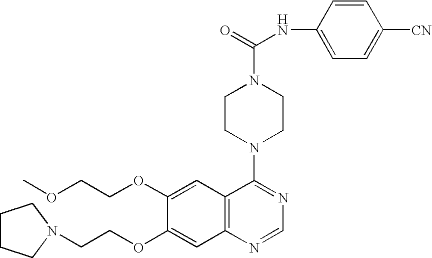
and all pharmaceutically acceptable isomers, salts, hydrates, solvates and prodrug derivatives thereof.

8. A pharmaceutical composition comprising an effective amount of a nitrogen-containing heterocyclic compound according to claim 1, or a pharmaceutically acceptable salt thereof, and a pharmaceutically acceptable diluent or carrier.

9. A method of inhibiting phosphorylation of PDGF receptor in a patient comprising administering a composition according to claim 8 to the patient.

10. A method for inhibiting abnormal cell growth and cell wandering in a patient and thereby preventing or treating a cell-proliferative disease, comprising the step of administering a composition according to claim 8 to the patient.

11. A method according to claim 10, wherein said cell-proliferative disease is selected from the group consisting of arteriosclerosis, vascular re-obstruction, restenosis, cancer and glomerulosclerosis.
